(19) United States
(12) Patent Application Publication
(10) Pub. No.: US 2025/0143585 A1
(43) Pub. Date: May 8, 2025

(54) SYSTEM, APPARATUS, AND METHOD FOR EVALUATING VASCULAR ENDOTHELIAL FUNCTION

(71) Applicant: Murata Manufacturing Co., Ltd., Nagaokakyo-shi (JP)

(72) Inventor: Toru SHIMUTA, Nagaokakyo-shi (JP)

(21) Appl. No.: 19/018,361

(22) Filed: Jan. 13, 2025

Related U.S. Application Data
(63) Continuation of application No. PCT/JP2023/030016, filed on Aug. 21, 2023.

(30) Foreign Application Priority Data
Sep. 9, 2022 (JP) 2022-143981

(51) Int. Cl. A61B 5/02; A61B 5/00; A61B 5/021; G16H 50/30

(52) U.S. Cl. CPC A61B 5/02007; A61B 5/0053; A61B 5/02116; A61B 5/02125; A61B 5/02141; A61B 5/6826; A61B 5/7239; A61B 5/742; G16H 50/30; A61B 2560/0462; A61B 2562/0233

(57) ABSTRACT

A pulse wave measurement unit is provided that generates a pulse wave signal based on a measurement result from a pulse wave sensor attached to a site farther from the heart than a pressurization site that is pressurized for vascular occlusion. A peripheral blood pressure index calculation unit calculates a peripheral blood pressure index related to steepness of a rise per beat of the pulse wave signal generated by the pulse wave measurement unit. A vascular endothelial function evaluation unit evaluates vascular endothelial function, based on a calculated value of the peripheral blood pressure index from a time point of vascular occlusion release until an evaluation duration elapses.

### CROSS REFERENCE TO RELATED APPLICATIONS

This application is a continuation of International Application No. PCT/JP2023/030016, filed Aug. 21, 2023, which claims priority to Japanese Patent Application No. 2022-143981, filed Sep. 9, 2022, the contents of each of which are hereby incorporated by reference in their entirety.

### TECHNICAL FIELD

The present disclosure relates to a system, apparatus, and method for evaluating vascular endothelial function.

### BACKGROUND

Vascular endothelial cells function to contract and relax blood vessel walls, facilitate the adhesion of inflammatory cells to blood vessel walls, regulate vascular permeability and modulate the coagulation-fibrinolytic system, and perform other functions. These functions of vascular endothelial cells (e.g., a vascular endothelial function) can deteriorate due to various lifestyle-related diseases such as hypertension, diabetes, dyslipidemia, and obesity. For example, the blood flow-dependent vasodilatory response test (flow-mediated dilatation, FMD) and the EndoPAT test are known methods for evaluating vascular endothelial function.

Currently, apparatuses for evaluating vascular endothelial function using a cuff sphygmomanometer are known, such as that described in Japanese Unexamined Patent Application Publication No. 2013-126487. This evaluation apparatus evaluates vascular endothelial function based on the pulse wave detected by a pressure sensor connected to the cuff of the cuff sphygmomanometer during any two periods among before, during, and after pressurization.

To evaluate vascular endothelial function, this known apparatus for evaluating vascular endothelial function uses pulse waves acquired by the pressure sensor connected to the cuff. This pulse wave acquired by the pressure sensor primarily provides information regarding large blood vessels and does not provide information regarding fine blood vessels such as arterioles and capillaries.

### SUMMARY OF THE INVENTION

In view of the foregoing, it is an object of the present disclosure to provide a system, apparatus, and method for evaluating vascular endothelial function involving arterioles and capillaries.

In an exemplary aspect, a system is provided for evaluating vascular endothelial function. The system includes a pulse wave measurement unit configured to generate a pulse wave signal based on a measurement result from a pulse wave sensor attached to a site farther from the heart than a pressurization site that is pressurized for vascular occlusion, an output unit, a peripheral blood pressure index calculation unit configured to calculate a peripheral blood pressure index related to steepness of a rise per beat of the pulse wave signal generated by the pulse wave measurement unit, and a vascular endothelial function evaluation unit configured to evaluate vascular endothelial function, based on a calculated value of the peripheral blood pressure index from a time point of vascular occlusion release until an evaluation duration elapses.

Another exemplary aspect of the present disclosure provides an apparatus for evaluating vascular endothelial function, including a pulse wave measurement device configured to generate a pulse wave signal based on a measurement result from a pulse wave sensor attached to a site farther from the heart than a pressurization site that is pressurized for vascular occlusion, and a control terminal configured to calculate a peripheral blood pressure index related to steepness of a rise per beat of the pulse wave signal generated by the pulse wave measurement device, evaluate vascular endothelial function based on a calculated value of the peripheral blood pressure index from a time point of vascular occlusion release until an evaluation duration elapses, and output an evaluation result.

Another exemplary aspect of the present disclosure provides a method for evaluating vascular endothelial function, including performing vascular occlusion by pressurizing a part of a body and subsequently releasing vascular occlusion, and acquiring a pulse wave signal of an arteriole or capillary at a site farther from the heart than a pressurized site from a time point of vascular occlusion release until a certain duration elapses, obtaining a temporal change in a peripheral blood pressure index related to steepness of a rise per beat of the acquired pulse wave signal, and evaluating vascular endothelial function based on the temporal change in the peripheral blood pressure index.

The peripheral blood pressure index includes information regarding blood flow in peripheral blood vessels such as arterioles and capillaries. Because vascular endothelial function is evaluated based on calculated values of the peripheral blood pressure index from the time point of vascular occlusion release until the evaluation duration elapses, vascular endothelial function including information regarding peripheral blood vessels is evaluated.

### BRIEF DESCRIPTION OF DRAWINGS

FIG. 1 is a block diagram of a system for evaluating vascular endothelial function according to a first exemplary embodiment.

FIG. 2A is a perspective view of a pulse wave measurement device, and FIG. 2B is a schematic diagram of a photoelectric pulse wave sensor and biological tissue in the state in which the pulse wave measurement device is worn on a finger.

FIG. 3 is a flowchart illustrating the procedure for a method for evaluating vascular endothelial function according to the first exemplary embodiment.

FIG. 4 is a graph illustrating an example of a pulse wave, velocity pulse wave, and acceleration pulse wave.

FIG. 5 is a graph illustrating an example of a pulse wave and acceleration pulse wave.

FIGS. 6A and 6B are graphs plotting the value of a peripheral blood pressure index “1/VE0.5” against the systolic blood pressure measured at the wrist. The peripheral blood pressure index “1/VE0.5” is obtained from the pulse wave measured when the height from the heart to the measurement site (finger) is changed, or when the area including the elbow on the same side as the finger serving as the measurement site is cooled while the measurement site is maintained at the same height as the chest.

FIGS. 7A and 7B are graphs plotting the value of a peripheral blood pressure index “a/S” against the systolic blood pressure measured at the wrist. The peripheral blood pressure index “a/S” is obtained from the pulse wave measured when the height from the heart to the measurement site (finger) is changed, or when the area including the elbow on the same side as the finger serving as the measurement site is cooled while the measurement site is maintained at the same height as the chest.

FIGS. 8A and 8B are graphs plotting the value of a peripheral blood pressure index “(a-b)/(a-d)” against the systolic blood pressure measured at the wrist. The peripheral blood pressure index “(a-b)/(a-d)” is obtained from the pulse wave measured when the height from the heart to the measurement site (finger) is changed, or when the area including the elbow on the same side as the finger serving as the measurement site is cooled while the measurement site is maintained at the same height as the chest.

FIG. 9 is a graph illustrating the pulse wave obtained from subject A whose blood pressure is within the normal range.

FIGS. 10A to 10F are graphs illustrating the temporal changes in the peripheral blood pressure indices obtained from the pulse wave illustrated in FIG. 9.

FIG. 11 is a graph illustrating the pulse wave obtained from another subject B whose blood pressure is within the normal range.

FIGS. 12A to 12F are graphs illustrating the temporal changes in the peripheral blood pressure indices obtained from the pulse wave illustrated in FIG. 11.

FIG. 13 is a graph illustrating the pulse wave obtained from subject C whose blood pressure exceeds the normal range.

FIGS. 14A, 14B, and 14C are graphs illustrating the temporal changes in the peripheral blood pressure indices obtained from the pulse wave illustrated in FIG. 13.

FIG. 15 is a graph illustrating the pulse wave measured with an extended vascular occlusion duration for subject A, whose pulse wave was measured as illustrated in FIG. 9.

FIGS. 16A to 16F are graphs illustrating the temporal changes in the peripheral blood pressure indices obtained from the pulse wave illustrated in FIG. 15.

FIG. 17 is a flowchart illustrating the procedure for a method for evaluating vascular endothelial function according to a second exemplary embodiment.

FIG. 18 is a flowchart illustrating the procedure for a method for evaluating vascular endothelial function according to a modification of the second exemplary embodiment.

FIGS. 19A and 19B are graphs illustrating the temporal changes in an amplitude S (FIG. 5) and the peripheral blood pressure index “a/S” obtained from the pulse wave of subject A illustrated in FIG. 15 according to a third exemplary embodiment.

FIG. 20 is a block diagram of a system for evaluating vascular endothelial function according to a fourth exemplary embodiment.

FIG. 21 is a schematic diagram of a user wearing a measurement device to evaluate vascular endothelial function using the system for evaluating vascular endothelial function according to the exemplary fourth embodiment.

### DETAILED DESCRIPTION OF EMBODIMENTS

### First Exemplary Embodiment

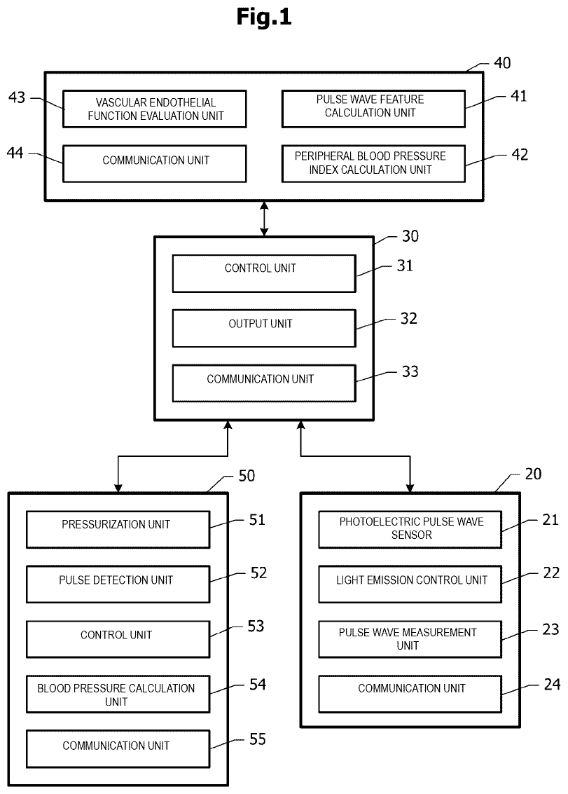

A system and method for evaluating vascular endothelial function according to a first exemplary embodiment will be described with reference to the drawings in FIGS. 1 to 14C.

FIG. 1 is a block diagram of the system for evaluating vascular endothelial function according to the first embodiment. The system for evaluating vascular endothelial function includes a pulse wave measurement device 20, a control terminal 30, a server 40, and a cuff sphygmomanometer 50. The pulse wave measurement device 20, the control terminal 30, the server 40, and the cuff sphygmomanometer 50 respectively include communication units 24, 33, 44, and 55. The pulse wave measurement device 20, the control terminal 30, the server 40, and the cuff sphygmomanometer 50 are configured for data communication with each other via these communication units. For example, wireless communication compliant with short-range wireless communication standards such as Bluetooth® is used for communication between the pulse wave measurement device 20 and the control terminal 30 and communication between the cuff sphygmomanometer 50 and the control terminal 30. For example, a wireless local area network (LAN) is used for communication between the control terminal 30 and the server 40.

The pulse wave measurement device 20 includes a photoelectric pulse wave sensor 21, a light emission control unit 22, a pulse wave measurement unit 23, and the communication unit 24. The photoelectric pulse wave sensor 21 includes a light-emitting element and a light-receiving element. The light output from the light-emitting element and transmitted through biological tissue is received by the light-receiving element, and the intensity of the received light is measured. The light emission control unit 22 is operable to control the light emission of the light-emitting element. The pulse wave measurement unit 23 is configured to generate a pulse wave signal based on the measured light intensity from the photoelectric pulse wave sensor 21.

The control terminal 30 includes a control unit 31, an output unit 32, and the communication unit 33. For example, a smartphone or similar device can be used as the control terminal 30. By installing an application program on a smartphone, the smartphone can be configured as the control terminal 30. Thus, it is generally noted that each of the components of control terminal 30 can be implemented as a processor or a microprocessor of a CPU configured to execute one or more software algorithms stored on electronic memory for implementing the algorithms described herein.

The control unit 31 is configured to receive pulse wave signals from the pulse wave measurement device 20 and to transfer the received pulse wave signals to the server 40. The control unit 31 is also configured to receive evaluation results regarding vascular endothelial function from the server 40 and output the evaluation results the output unit 32. The output unit 32 includes, for example, a display device for displaying images. The display device displays the evaluation results regarding vascular endothelial function in the form of images or text.

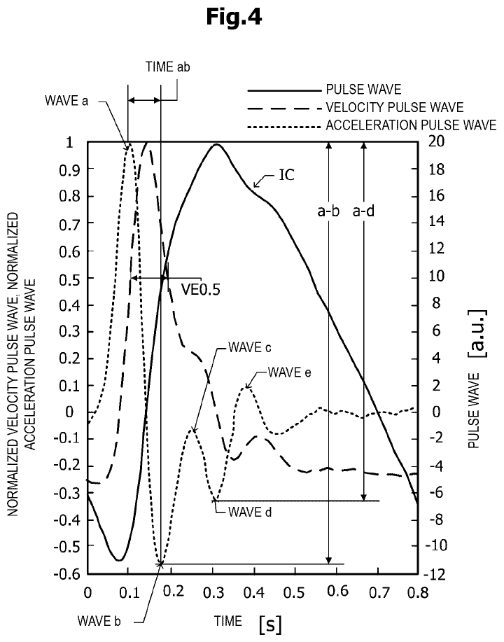

The server 40 includes a pulse wave feature calculation unit 41, a peripheral blood pressure index calculation unit 42, a vascular endothelial function evaluation unit 43, and the communication unit 44. The pulse wave feature calculation unit 41 is configured to calculate various features of the waveform (hereinafter also referred to simply as “pulse wave”) of the pulse wave signal received from the control terminal 30. The peripheral blood pressure index calculation unit 42 is configured to calculate peripheral blood pressure indices related to the steepness of the rise of the pulse wave per beat, based on the pulse wave features. The peripheral blood pressure indices can be used as indices of the magnitude of peripheral blood pressure. The peripheral blood pressure indices will be described in detail later with reference to the drawings in FIGS. 4 to 8B.

The vascular endothelial function evaluation unit 43 is configured to evaluate vascular endothelial function based on temporal changes in the peripheral blood pressure indices and to transmit the evaluation results to the control terminal 30. The vascular endothelial function evaluation unit 43 is also configured to store the evaluation results.

The cuff sphygmomanometer 50 includes a pressurization unit 51, a pulse detection unit 52, a control unit 53, a blood pressure calculation unit 54, and the communication unit 55. For example, a cuff automatic electronic sphygmomanometer can be used as the cuff sphygmomanometer 50. The control unit 53 is configured to receive instructions from the control terminal 30 and control the pressurization by the pressurization unit 51. The pressurization unit 51 includes a cuff. With the cuff wrapped around the user's upper arm, vascular occlusion can be initiated or released through pressurization control. The pulse detection unit 52 is configured to detect the pulse at the site where the cuff is wrapped. The blood pressure calculation unit 54 is configured to calculate blood pressure based on the pressure applied to the cuff and the detection result from the pulse detection unit 52. At least the control unit 53 and the blood pressure calculation unit 54 can be implemented as a processor or a microprocessor of a CPU configured to execute one or more software algorithms stored on electronic memory for implementing the algorithms described herein.

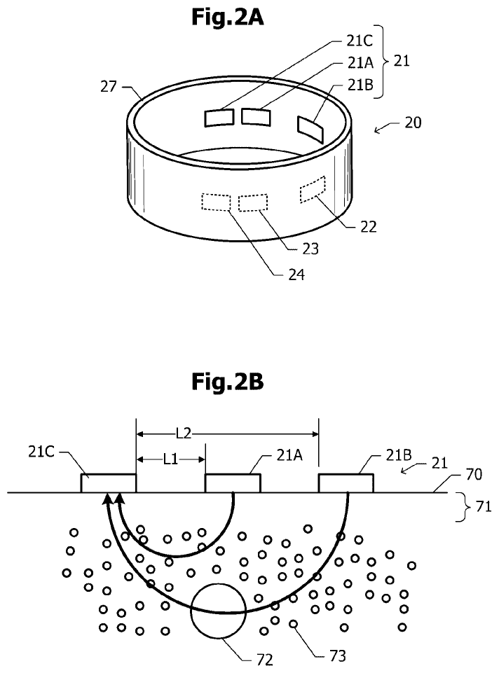

FIG. 2A is a perspective view of the pulse wave

measurement device 20. In the first embodiment, a ring-type device configured to be worn on the user's finger is used as the pulse wave measurement device 20. The photoelectric pulse wave sensor 21 includes two light-emitting elements 21A and 21B and one light-receiving element 21C. The two light-emitting elements 21A and 21B and the one light-receiving element 21C are disposed on the inner surface of a ring-shaped wearable member 27. It is noted that only one of the light-emitting element 21A and 21B may be included in the configuration in an alternative aspect.

When the wearable member 27 is worn on the finger, the light-emitting elements 21A and 21B is configured to emit light toward the finger. The light-receiving element 21C is attached at the location at which light reflected by the biological tissue inside the finger or transmitted through the biological tissue enters.

Additionally, the light emission control unit 22, the pulse wave measurement unit 23, and the communication unit 24 are integrated into the wearable member 27. The light emission control unit 22, the pulse wave measurement unit 23, and the communication unit 24 may be configured as a single integrated circuit in an exemplary aspect.

FIG. 2B is a schematic diagram of the photoelectric pulse wave sensor 21 and biological tissue when the pulse wave measurement device 20 is worn on the finger. The light-emitting elements 21A and 21B and the light-receiving element 21C are in contact with a user's body surface 70. The light-emitting elements 21A and 21B are configured to emit measurement light toward the body surface 70. The emitted light is absorbed, reflected, and/or scattered (hereinafter sometimes simply referred to as “reflected”) by an epidermal region 71, arterioles 72, and capillaries 73 within the body surface 70. A portion of the light that passes through biological tissue such as the epidermal region 71, the arterioles 72, and the capillaries 73 enters the light-receiving element 21C.

It is known that the arterioles 72 are fine blood vessels with diameters, for example, greater than or equal to 20 μm and smaller than or equal to 200 μm, located between arteries and the capillaries 73. Multiple capillaries 73 branch off from each arteriole 72. The capillaries 73 are fine blood vessels with diameters of, for example, approximately 10 μm, connecting arteries and veins. Multiple capillaries 73 are distributed in a region shallower than the region in which the arterioles 72 are distributed. The blood in arteries contains hemoglobin that has the characteristic of absorbing measurement light. The blood flow rate fluctuates with heartbeats, and the amount of light absorbed varies with the fluctuations in the blood flow rate. As a result, the intensity of the light received by the light-receiving element 21C changes with heartbeats.

As the light-emitting element 21A, an element that outputs light in the wavelength range, for example, from blue to yellow-green (e.g., a wavelength range greater than or equal to 450 nm and smaller than or equal to 570 nm), preferably in the wavelength range greater than or equal to 500 nm smaller than or equal to 550 nm, is used. The light-emitting element 21B outputs light in the wavelength range from red to near-infrared, preferably in the wavelength range greater than or equal to 750 nm and smaller than or equal to 950 nm. For example, light-emitting diodes (LEDs) or vertical-cavity surface-emitting lasers (VCSELs) are used as the light-emitting elements 21A and 21B. For example, a photodiode (PD) or phototransistor is used as the light-receiving element 21C.

It is understood that biological tissue strongly absorbs light in the wavelength range from blue to yellow-green. For this reason, pulse waves acquired using light in the wavelength range from blue to yellow-green reflect information from a shallow region beneath the skin surface, particularly a region shallower than where the arterioles 72 are distributed. In this specific region, the capillaries 73 are primarily distributed. The arrow from the light-emitting element 21A to the light-receiving element 21C illustrated in FIG. 2B does not represent the path of light propagation, but indicates that the light output from the light-emitting element 21A enters the light-receiving element 21C after passing through the epidermal region 71 and the region in which the capillaries 73 are primarily distributed. To ensure that information from the region shallower than the region in which the arterioles 72 are distributed, where the capillaries 73 are primarily distributed, is strongly reflected in the acquired pulse waves, a distance L1 between the light-emitting element 21A and the light-receiving element 21C is preferably short. For example, the distance L1 is preferably greater than or equal to 1 mm and smaller than or equal to 3 mm.

Light with a wavelength shorter than 450 nm can damage biological tissue. To avoid damaging biological tissue, the wavelength of light used for pulse wave measurement is preferably 450 nm or greater in an exemplary aspect.

Biological tissue absorbs light in the wavelength range from red to near-infrared less than light in the wavelength range from blue to yellow-green. As a result, pulse waves acquired using light in the wavelength range from red to near-infrared reflect information from deeper regions beneath the skin surface.

For example, the pulse waves reflect information from the regions in which the capillaries 73 and the arterioles 72 are distributed. The arrow from the light-emitting element 21B to the light-receiving element 21C illustrated in FIG. 2B does not represent the path of light propagation, but indicates that the light output from the light-emitting element 21B enters the light-receiving element 21C after passing through the region in which the arterioles 72 are distributed as well as the region in which the capillaries 73 are distributed. To ensure that information from the regions in which the arterioles 72 and the capillaries 73 are distributed is strongly reflected in the acquired pulse waves, a distance L2 between the light-emitting element 21B and the light-receiving element 21C is preferably greater than or equal to 5 mm and smaller than or equal to 20 mm.

In the wavelength range longer than 950 nm, the absorbance of hemoglobin decreases. For this reason, light in the wavelength range smaller than or equal to 950 nm is preferably used for pulse wave signal acquisition.

In the example illustrated in FIG. 2B, a single light-receiving element 21C is provided for two light-emitting elements 21A and 21B. However, one light-receiving element may be provided for one light-emitting element 21A, and another light-receiving element may be provided for the other light-emitting element 21B.

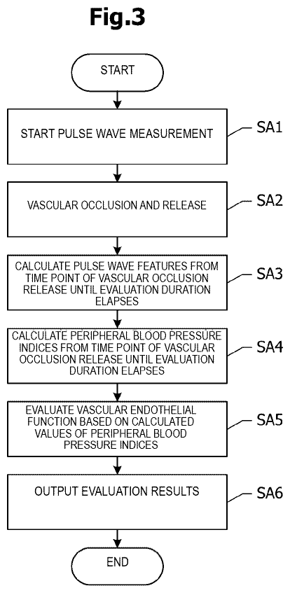

FIG. 3 is a flowchart illustrating the procedure for the method for evaluating vascular endothelial function according to the first exemplary embodiment. Prior to evaluation, the user wraps the cuff of the cuff sphygmomanometer 50 around either one of the upper arms or one of the wrists. In addition, the user wears the pulse wave measurement device 20 (e.g., as shown in FIG. 2A) on the index finger of the same arm around which the cuff is wrapped. The pulse wave measurement device 20 may be worn on any finger other than the index finger and may be worn on the fingertip.

First, the control terminal 30 (FIG. 1) is configured to control the pulse wave measurement device 20 (FIG. 1) to start pulse wave measurement (step SA1). The measured pulse wave is transmitted to the server 40 (FIG. 1). The control terminal 30 then controls the cuff sphygmomanometer 50 (FIG. 1) to initiate vascular occlusion and release vascular occlusion after a certain duration elapses (step SA2).

The pulse wave feature calculation unit 41 of the server 40 is then configured to calculate the pulse wave features of the pulse wave waveform for each beat, from the time point of vascular occlusion release until a predetermined evaluation duration elapses (step SA3). The length of the evaluation duration is predetermined. Subsequently, the peripheral blood pressure index calculation unit 42 of the server 40 is configured to calculate the peripheral blood pressure indices for each beat of the pulse wave, based on the calculated pulse wave feature values from the time point of vascular occlusion release until the evaluation duration elapses (step SA4). This operation determines temporal changes in the peripheral blood pressure indices over the evaluation duration.

Next, the vascular endothelial function evaluation unit 43 of the server 40 is configured to evaluate vascular endothelial function based on the multiple calculated values of the peripheral blood pressure indices (step SA5). The vascular endothelial function evaluation unit 43 transmits the evaluation result to the control terminal 30. The control unit 31 of the control terminal 30 outputs the evaluation result received from the server 40 to the output unit 32 (step SA6). Vascular endothelial function is evaluated, for example, on a five-point scale ranging from level 1 to level 5.

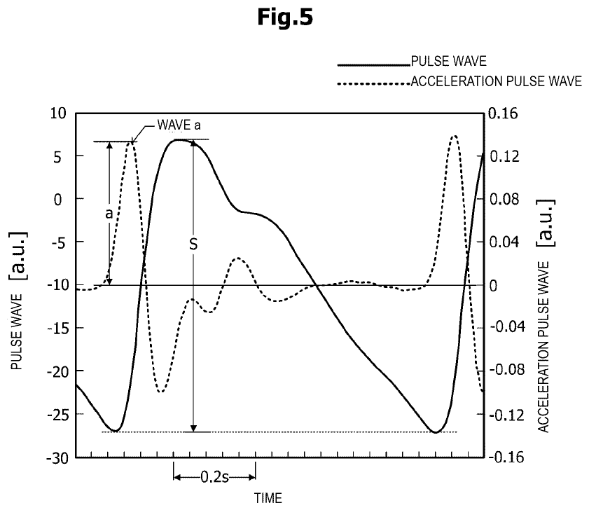

Next, various pulse wave features are described with reference to FIGS. 4 and 5.

FIG. 4 is a graph illustrating an example of a pulse wave, velocity pulse wave, and acceleration pulse wave.

The pulse wave feature calculation unit 41 (FIG. 1) of the server 40 calculates the first and second derivatives of the pulse wave. The waveform obtained by calculating the first derivative of the pulse wave is referred to as the velocity pulse wave, and the waveform obtained by calculating the second derivative of the pulse wave is referred to as the acceleration pulse wave. For example, the velocity pulse wave is obtained by numerically differentiating, by the time intervals corresponding to the sampling rate, the intensity of the pulse wave discretely distributed at time intervals corresponding to the sampling rate. The acceleration pulse wave is obtained by numerically differentiating the magnitude of the velocity pulse wave.

The horizontal axis in FIG. 4 represents time in units of [s], the left vertical axis represents the magnitudes of the normalized velocity pulse wave and the magnitude of the normalized acceleration pulse wave, both normalized to have a maximum value of 1, and the right vertical axis represents the magnitude of the pulse wave in arbitrary units. The solid, long-dashed, and short-dashed lines in the graph illustrated in FIG. 4 respectively represents pulse wave, velocity pulse wave, and acceleration pulse wave. Typically, five peaks appear in an acceleration pulse wave within one beat. The first, second, third, fourth, and fifth peaks within one beat are respectively referred to as wave a, wave b, wave c, wave d, and wave e.

As further shown, the full width at half maximum of the first upward peak of the velocity pulse wave is denoted as “VE0. 5”. The difference between the peak value of wave a and the peak value of wave b is denoted as “a-b”, and the difference between the peak value of wave a and the peak value of wave d is denoted as “a-d”. A depression, referred to as a notch IC, appears shortly after the maximum peak of the pulse wave.

FIG. 5 is a graph illustrating an example of a pulse wave and acceleration pulse wave. The horizontal axis represents time, the left vertical axis represents the magnitude of the pulse wave in arbitrary units, and the right vertical axis represents the magnitude of the acceleration pulse wave in arbitrary units. The five tick marks on the horizontal axis together correspond to 0.2 s. The peak value of wave a in the acceleration pulse wave is denoted as “a”, and the amplitude of the pulse wave is denoted as “S”. The amplitude S of the pulse wave corresponds to the difference between the minimum and maximum values after the waveform is adjusted so that the minimum values of two consecutive beats of the pulse wave has the same magnitude.

Next, the peripheral blood pressure indices are described.

For purposes of this disclosure, “peripheral blood pressure” is defined as the blood pressure in peripheral arterioles and capillaries. Peripheral blood pressure is sometimes used to refer to wrist or ankle blood pressure measured using a cuff sphygmomanometer, but wrist or ankle blood pressure is measured on large arteries (such as the radial artery). As such, wrist or ankle blood pressure differs from the peripheral blood pressure as described in this disclosure. In the order from large arteries to arterioles and then to capillaries, the blood pressure in the blood vessels decreases. The degree to which blood pressure decreases depends on the measurement site, the individual's vascular condition (for example, presence or absence of arteriosclerosis), mental condition (for example, autonomic nerve condition), environment (for example, temperature, presence or absence of noise), clothing, and other factors.

Among the pulse wave features, the indices effective for determining peripheral blood pressure are used as the peripheral blood pressure indices. The peripheral blood pressure indices are considered to have the following characteristics.

First, when blood vessels are healthy and under the condition that vascular resistance is unchanged, the peripheral blood pressure indices positively correlate with blood pressure at the upper arm or wrist. Second, when blood vessels constrict due to cooling the area near the measurement site, the peripheral blood pressure indices decrease. When blood vessels constrict, peripheral vascular resistance increases, and blood pressure at the upper arm or wrist may increase in some cases.

The following three pulse wave features reflect the

two characteristics of the peripheral blood pressure indices:
The reciprocal of the full width at half maximum “VE0. 5” (hereafter labeled “1/(VE0.5)”)
Ratio of a peak value a of wave a of the acceleration pulse wave to the amplitude S of the pulse wave (hereinafter labeled “a/S”)
The ratio of the difference “a-b” between the peak value of wave a and the peak value of wave b of the acceleration pulse wave to the difference “a-d” between the peak value of wave a and the peak value of wave d (hereinafter labeled “(a-b)/(a-d)”).

For purposes of this disclosure, these features of the pulse wave waveform are referred to as the “peripheral blood pressure indices.” These peripheral blood pressure indices are related to the steepness of the rise of the pulse wave.

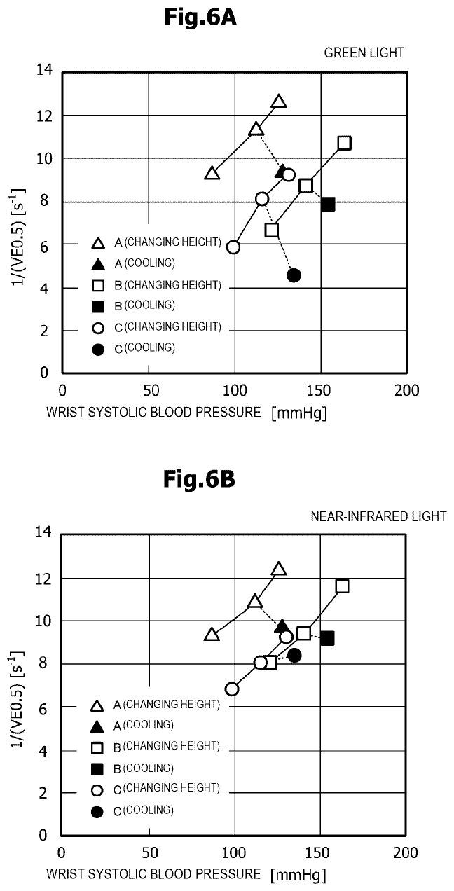

FIGS. 6A and 6B are graphs plotting the value of the peripheral blood pressure index “1/VE0.5” against the systolic blood pressure measured at the wrist. The peripheral blood pressure index “1/VE0.5” is obtained from the pulse wave measured when the height from the heart to the measurement site (e.g., a finger) is changed, or when the area including the elbow on the same side as the finger serving as the measurement site is cooled while the measurement site is maintained at the same height as the chest. FIGS. 6A and 6B respectively illustrate the measurement results when the pulse wave is measured using green light output from the light-emitting element 21A (FIG. 2A) and near-infrared light output from the light-emitting element 21B (FIG. 2A). The pulse wave measured using green light primarily reflects variations in blood flow in the capillaries 73 (FIG. 2B). The pulse wave measured using near-infrared light reflects variations in blood flow in the capillaries 73 and the arterioles 72 (FIG. 2B).

The horizontal axis of the graphs in FIGS. 6A and 6B represents systolic blood pressure at the wrist in units of [mmHg], and the vertical axis represents the peripheral blood pressure index “1/(VE0.5)” in units of [s⁻¹]. In each graph, the results of measurements performed on three subjects A, B, and C are respectively indicated by triangle, square, and circle symbols. The three hollow symbols illustrated for each subject indicate the values of the peripheral blood pressure index “1/VE0.5” obtained from the pulse wave acquired when the measurement site (e.g., the finger) is positioned at the height of the navel, chest, and forehead. The value of the peripheral blood pressure index “1/VE0.5” decreases in the order of the navel, chest, and forehead as the height of the measurement site is raised. The black-filled symbol illustrated for each subject indicates the value of the peripheral blood pressure index “1/VE0.5” obtained from the pulse wave acquired when the measurement site is positioned at the height of the chest while the area including the elbow is cooled.

Although the degree varies among the subjects, it is shown that the peripheral blood pressure index “1/VE0.5” generally indicates a positive correlation with systolic blood pressure at the wrist as the height of the measurement site is changed. Further, although there is an exception, it is also shown that the peripheral blood pressure index “1/VE0.5” decreases when blood vessels constrict due to cooling the area near the measurement site. This pattern of changes is consistent with the assumed characteristics of the peripheral blood pressure indices. Therefore, the peripheral blood pressure index “1/VE0.5” is considered an effective index for estimating peripheral blood pressure.

The results illustrated in FIGS. 6A and 6B indicate that green light is preferable to near-infrared light for measuring the peripheral blood pressure index “1/VE0.5.” As an alternative index to the peripheral blood pressure index “1/VE0.5”, the reciprocal of the parameter representing the width of the largest peak of the velocity pulse wave may be used. Alternatively, the parameter representing the width of the largest peak of the velocity pulse wave raised to a negative exponent may be used. More generally, as the peripheral blood pressure index, a function with a parameter representing the width of the largest peak of the velocity pulse wave as a variable may be used, where the value of the function decreases as the width of the peak increases.

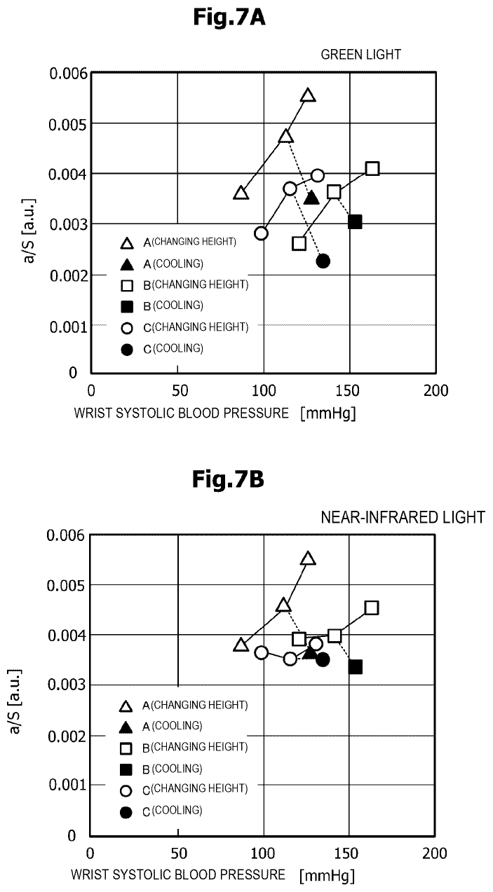

FIGS. 7A and 7B are graphs plotting the value of the peripheral blood pressure index “a/S” against the systolic blood pressure measured at the wrist. The peripheral blood pressure index “a/S” is obtained from the pulse wave measured when the height from the heart to the measurement site (finger) is changed, or when the area including the elbow on the same side as the finger serving as the measurement site is cooled while the measurement site is maintained at the same height as the chest. FIGS. 7A and 7B respectively illustrate the measurement results when the pulse wave is measured using green light and near-infrared light.

The horizontal axis of the graphs in FIGS. 7A and 7B represents systolic blood pressure at the wrist in units of [mmHg], and the vertical axis represents the peripheral blood pressure index “a/S” in arbitrary units. The meaning of each symbol in FIGS. 7A and 7B is identical to the meaning of each symbol in the graphs illustrated in FIGS. 6A and 6B as described above.

The measurement results illustrated in FIGS. 7A and 7B indicate similar tendencies to the measurement results illustrated in FIGS. 6A and 6B. Therefore, the peripheral blood pressure index “a/S” is considered an effective index for estimating peripheral blood pressure. The results illustrated in FIGS. 7A and 7B indicate that green light is preferable to near-infrared light for measuring the peripheral blood pressure index “a/S.”

Instead of the peripheral blood pressure index “a/S”, the product of the peak value a of wave a of the acceleration pulse wave raised to a positive exponent and the amplitude S of the pulse wave raised to a negative exponent may be used as the peripheral blood pressure index. Alternatively, the peripheral blood pressure index may be calculated based on information regarding the peak value of wave a of the acceleration pulse wave and the amplitude of the pulse wave signal. For example, as the peripheral blood pressure index, a function with the peak value a and the amplitude S as variables may be used, where the value of the function increases as the peak value a increases, and the value of the function decreases as the amplitude S increases.

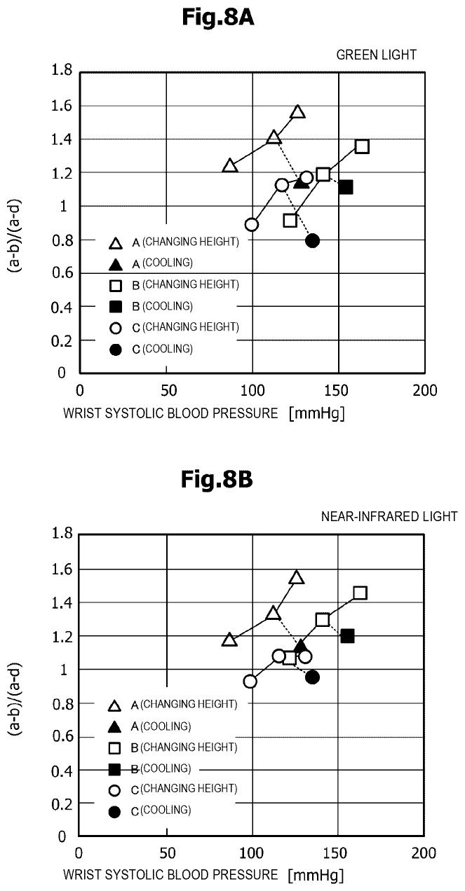

FIGS. 8A and 8B are graphs plotting the value of the peripheral blood pressure index “(a-b)/(a-d)” against the systolic blood pressure measured at the wrist. The peripheral blood pressure index “(a-b)/(a-d)” is obtained from the pulse wave measured when the height from the heart to the measurement site (e.g., the finger) is changed, or when the area including the elbow on the same side as the finger serving as the measurement site is cooled while the measurement site is maintained at the same height as the chest. FIGS. 8A and 8B respectively illustrate the measurement results when the pulse wave is measured using green light and near-infrared light.

The horizontal axis of the graphs in FIGS. 8A and 8B represents systolic blood pressure at the wrist in units of [mmHg], and the vertical axis represents the peripheral blood pressure index “(a-b)/(a-d)”. The meaning of each symbol in FIGS. 8A and 8B is identical to the meaning of each symbol in the graphs illustrated in FIGS. 6A and 6B as described above.

The measurement results illustrated in FIGS. 8A and 8B indicate similar tendencies to the measurement results illustrated in FIGS. 6A and 6B. Therefore, the peripheral blood pressure index “(a-b)/(a-d)” is considered an effective index for estimating peripheral blood pressure.

Instead of the peripheral blood pressure index “(a-b)/(a-d)”, the peripheral blood pressure index may be calculated based on information regarding the difference between the peak value of wave a and the peak value of wave b of the acceleration pulse wave and the difference between the peak value of wave a and the peak value of wave d of the acceleration pulse wave. For example, as the peripheral blood pressure index, a function with the difference (a-b) between the peak value of wave a and the peak value of wave b and the difference (a-d) between the peak value of wave a and the peak value of wave d as variables may be used, where the value of the function increases as the value of the difference (a-b) increases, and the value of the function decreases as the value of the difference (a-d) increases.

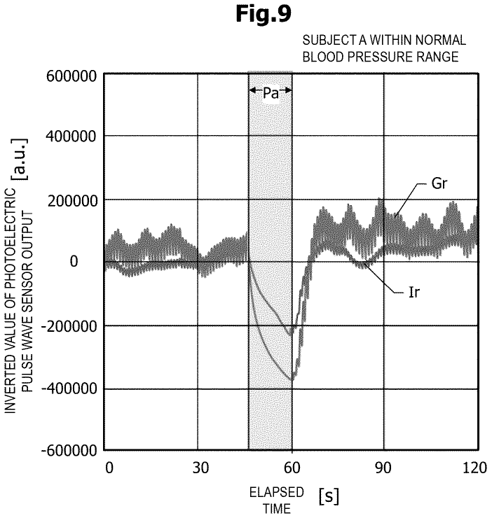

Next, the method for evaluating vascular endothelial function using the peripheral blood pressure indices is described with reference to the drawings in FIGS. 9 to 14C.

FIG. 9 is a graph illustrating the pulse wave obtained from subject A whose blood pressure is within the normal range. The horizontal axis represents the elapsed time from the start of pulse wave measurement in units of [s], and the vertical axis represents the photoelectric pulse wave (the value obtained by inverting the output from the photoelectric pulse wave sensor 21 (FIG. 1)) in arbitrary units. Solid lines Gr and Ir in the graph in FIG. 9 respectively illustrate the measurement results when the pulse wave is measured using green light and near-infrared light. Subject A's systolic blood pressure was 115 mmHg, diastolic blood pressure was 76 mmHg, and pulse rate was 65 bpm.

Approximately 30 seconds after the start of pulse wave measurement, pressurization with the cuff sphygmomanometer 50 was initiated. It takes approximately ten seconds plus several seconds from the start of pressurization to the start of vascular occlusion. Once vascular occlusion starts, the pulse wave can no longer be acquired. Approximately 60 seconds after the start of pulse wave measurement, the pressure in the cuff decreases, allowing the pulse wave to be detected. The period during which vascular occlusion is ongoing is labeled Pa. The vascular occlusion period is shaded light gray in FIG. 9. The measurement was terminated when approximately 120 seconds had elapsed from the start of the measurement.

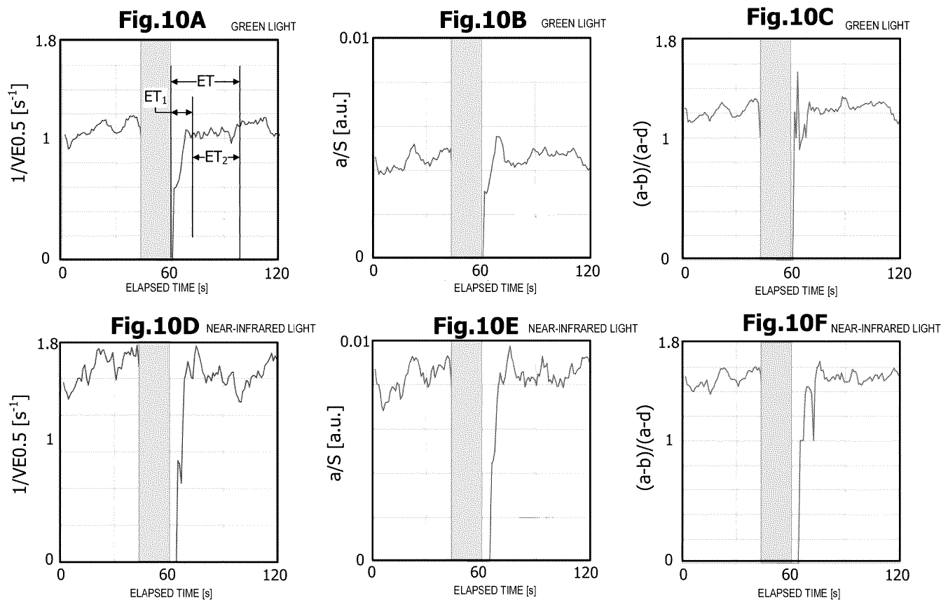

The graphs in FIGS. 10A to 10F illustrate the temporal changes in the peripheral blood pressure indices. The horizontal axis of these graphs represents the elapsed time in units of [s]. The vertical axis of the graphs in FIGS. 10A and 10D represents the peripheral blood pressure index “1/VE0.5” in units of [s⁻¹]. The vertical axis of the graphs in FIGS. 10B and 10E represents the peripheral blood pressure index “a/s” in arbitrary units. The vertical axis of the graphs in FIGS. 10C and 10F represents the peripheral blood pressure index “(a-b)/(a-d)”. The graphs in FIGS. 10A to 10C illustrate the peripheral blood pressure indices obtained from the pulse wave measured using green light. The graphs in FIGS. 10D to 10F illustrate the peripheral blood pressure indices obtained from the pulse wave measured using near-infrared light.

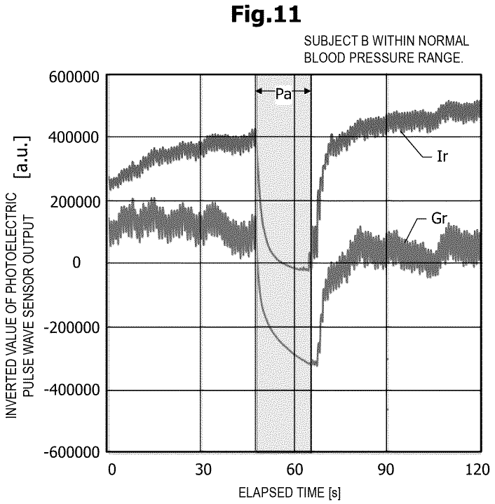

FIG. 11 is a graph illustrating the pulse wave obtained from another subject B whose blood pressure is within the normal range. The horizontal axis represents the elapsed time from the start of pulse wave measurement in units of [s], and the vertical axis represents the photoelectric pulse wave (the value obtained by inverting the output from the photoelectric pulse wave sensor 21 (FIG. 1)) in arbitrary units. Solid lines Gr and Ir in the graph in FIG. 11 respectively illustrate the measurement results when the pulse wave is measured using green light and near-infrared light. Subject B's systolic blood pressure was 104 mmHg, diastolic blood pressure was 76 mmHg, and pulse rate was 66 bpm.

The period of pressurization using the cuff sphygmomanometer 50 from the start to the end of pulse wave measurement is identical to that illustrated in FIG. 9. The period during which vascular occlusion is ongoing is labeled Pa. The vascular occlusion period is shaded light gray in FIG. 11.

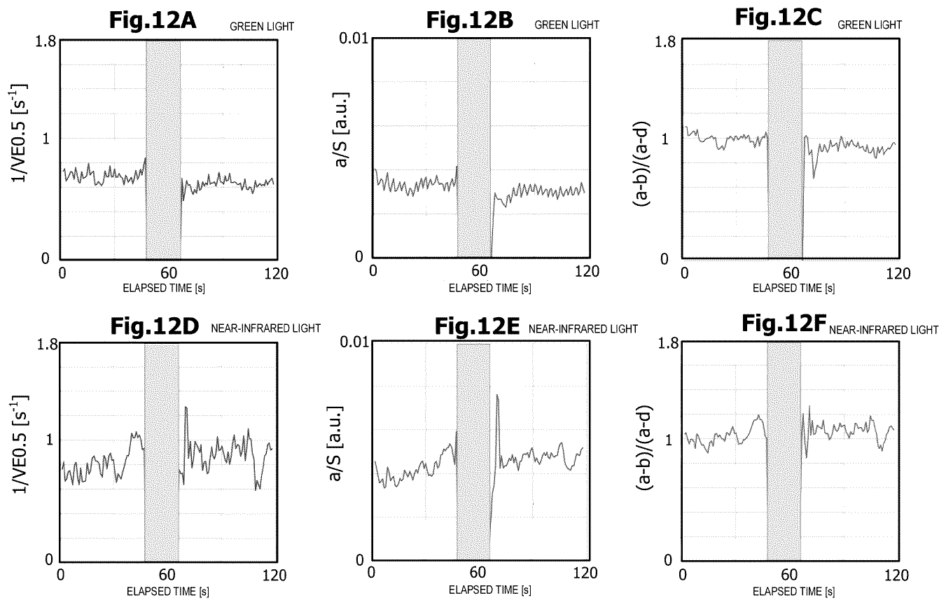

The graphs in FIGS. 12A to 12F illustrate the temporal changes in the peripheral blood pressure indices. The horizontal axis of these graphs represents the elapsed time in units of [s]. The vertical axis of the graphs in FIGS. 12A and 12D represents the peripheral blood pressure index “1/VE0.5” in units of [s⁻¹]. The vertical axis of the graphs in FIGS. 12B and 12E represents the peripheral blood pressure index “a/S” in arbitrary units. The vertical axis of the graphs in FIGS. 12C and 12F represents the peripheral blood pressure index “(a-b)/(a-d)”. The graphs in FIGS. 12A to 12C illustrate the peripheral blood pressure indices obtained from the pulse wave measured using green light. The graphs in FIGS. 12D to 12F illustrate the peripheral blood pressure indices obtained from the pulse wave measured using near-infrared light.

Both the peripheral blood pressure indices calculated from the pulse wave measured using green light and near-infrared light for subject A (the graphs in FIGS. 10A to 10F) and the peripheral blood pressure indices calculated from the pulse wave measured using green light for subject B (the graphs in FIGS. 12A, 12B, and 12C) decreased immediately after the release of vascular occlusion, compared to before the start of vascular occlusion. In this aspect, approximately 10 seconds after the release of vascular occlusion, the peripheral blood pressure indices returned to the values from before the start of vascular occlusion. The decrease in the peripheral blood pressure indices of subject A is more pronounced than the decrease in the peripheral blood pressure indices of subject B. Apparent decreases immediately after the release of vascular occlusion are not observed in the peripheral blood pressure indices calculated from the pulse wave measured using near-infrared light for subject B, as shown in the graphs in FIGS. 12D, 12E, and 12F.

The mechanism by which the peripheral blood pressure indices decrease immediately after the release of vascular occlusion can be considered as follows. When vascular endothelial function is normal, blood vessels dilate due to vascular endothelial function upon the release of vascular occlusion. The blood entering large blood vessels is used to dilate the large blood vessels, thereby suppressing the rapid inflow of blood to the downstream capillaries. As a result, the increase in the peripheral blood pressure indices is suppressed for approximately 10 seconds after the time point of vascular occlusion release.

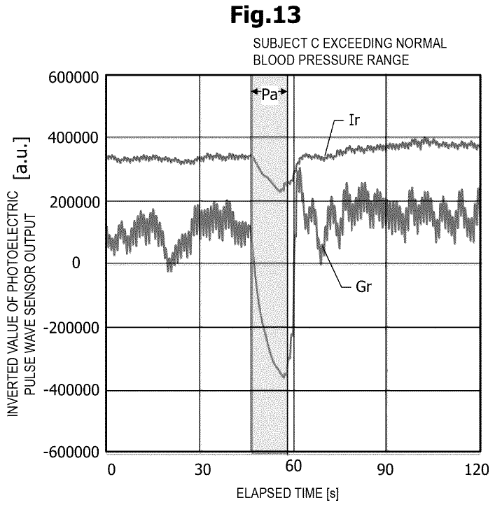

FIG. 13 is a graph illustrating the pulse wave obtained from subject C whose blood pressure exceeds the normal range. This subject is presumed to have impaired vascular endothelial function. The horizontal axis of the graph illustrated in FIG. 13 represents the elapsed time from the start of pulse wave measurement in units of [s], and the vertical axis represents the photoelectric pulse wave (the value obtained by inverting the output from the photoelectric pulse wave sensor 21 (FIG. 1)) in arbitrary units. Solid lines Gr and Ir in the graph in FIG. 13 respectively illustrate the measurement results when the pulse wave is measured using green light and near-infrared light. Subject C's systolic blood pressure was 164 mmHg, diastolic blood pressure was 104 mmHg, and pulse rate was 59 bpm.

The period of pressurization using the cuff sphygmomanometer 50 from the start to the end of pulse wave measurement is identical to that illustrated in FIG. 9. The period during which vascular occlusion is ongoing is labeled Pa. The vascular occlusion period is shaded light gray in FIG. 13.

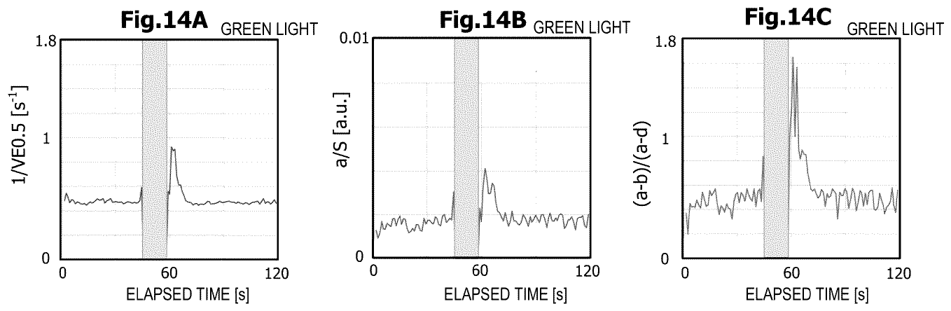

The graphs in FIGS. 14A, 14B, and 14C illustrate the temporal changes in the peripheral blood pressure indices. The horizontal axis of these graphs represents the elapsed time in units of [s]. The vertical axis of the graph in FIG. 14A represents the peripheral blood pressure index “1/VE0.5” in units of [s⁻¹]. The vertical axis of the graph in FIG. 14B represents the peripheral blood pressure index “a/S” in arbitrary units. The vertical axis of the graph in FIG. 14C represents the peripheral blood pressure index “(a-b)/(a-d)”. The graphs in FIGS. 14A to 14C illustrate the peripheral blood pressure indices obtained from the pulse wave measured using green light. Clear pulse wave features could not be calculated from the pulse wave acquired using near-infrared light.

Each peripheral blood pressure index increased immediately after the release of vascular occlusion compared to before the start of vascular occlusion. Approximately 10 seconds after the release of vascular occlusion, the peripheral blood pressure indices returned to the values from before the start of vascular occlusion. The mechanism by which the peripheral blood pressure indices increase immediately after the release of vascular occlusion can be considered as follows.

When vascular endothelial function is impaired, blood vessels do not dilate sufficiently upon the release of vascular occlusion. It is considered that the blood entering large blood vessels flows directly into the downstream arterioles and capillaries, thereby increasing the peripheral blood pressure indices.

As illustrated in the graphs in the drawings from FIGS. 9 to 14C, it is shown that the temporal changes in the peripheral blood pressure indices from the time point of vascular occlusion release until a certain duration has elapsed differs between individuals with normal vascular endothelial function and individuals with impaired vascular endothelial function. In the first embodiment, vascular endothelial function is evaluated using the differences in the patterns of the peripheral blood pressure indices after the release of vascular occlusion.

Next, the method for evaluating vascular endothelial function is described. The peripheral blood pressure indices are calculated based on the pulse wave from the time point of vascular occlusion release until a certain period (hereinafter referred to as an evaluation duration ET) elapses. FIG. 10A illustrates an example of the evaluation duration ET. The evaluation duration is divided into an anterior period ET₁ and a posterior period ET₂, and a mean value M₁ of the peripheral blood pressure index for the anterior period ET₁ and a mean value M₂ of the peripheral blood pressure index for the posterior period ET₂ are calculated.

Vascular endothelial function is evaluated based on the mean value M₁ of the peripheral blood pressure index for the anterior period ET₁ and the mean value M₂ of the peripheral blood pressure index for the posterior period ET². For example, it is assumed that vascular endothelial function becomes impaired as the value obtained by dividing the mean value M₁ of the peripheral blood pressure index for the anterior period ET₁ by the mean value M₂ of the peripheral blood pressure index for the posterior period ET₂ increases (in this disclosure, M₁/M₂ is referred to as a vascular endothelial function evaluation index). Vascular endothelial function may be evaluated on the five-point scale based on the magnitude of the vascular endothelial function evaluation index M₁/M₂. Alternatively, vascular endothelial function may be evaluated based on the difference between the mean values M₁ and M₂.

Comparing subjects A, B, and C with respect to the peripheral blood pressure indices before vascular occlusion, the peripheral blood pressure indices of subject A are the highest, and the peripheral blood pressure indices of subject C are the lowest. In comparison of the magnitude of the vascular endothelial function evaluation index M₁/M₂, the vascular endothelial function evaluation index M₁/M₂ of subject A is the smallest, and the vascular endothelial function evaluation index M₁/M₂ of subject C is the largest. It can be considered that subjects with lower vascular endothelial function tend to have lower peripheral blood pressure indices before vascular occlusion.

In an example, the evaluation duration ET can be set to 40 seconds and divided into the anterior period ET₁ and the posterior period ET₂ at the time point when 10 seconds have elapsed from the time point of vascular occlusion release. Moreover, the evaluation duration ET and the time point at which the anterior period ET₁ and the posterior period ET₂ are divided can be determined with reference to data obtained from an adequately large number of subjects.

For individuals with blood pressure higher than the normal range, pulse wave features are sometimes not calculated from the pulse wave acquired using near-infrared light, as described with reference to FIG. 13. To stably calculate pulse wave features for all individuals including individuals with blood pressure higher than the normal range, pulse waves are preferably acquired using light in the wavelength range from blue to yellow-green.

Next, advantageous effects of the first exemplary embodiment are described.

In the first embodiment, vascular endothelial function is evaluated by performing vascular occlusion on the vascular occlusion site in the upstream arteries through pressurization and measuring peripheral blood pressure in the downstream arterioles and capillaries. As a result, vascular endothelial function from the vascular occlusion site to the arterioles and capillaries is evaluated.

Next, a modification of the first embodiment is described.

In the first embodiment, the photoelectric pulse wave measured by the photoelectric pulse wave sensor 21 (FIG. 1) is used as the pulse wave for calculating the peripheral blood pressure indices. However, pulse waves measured by other sensors may also be used. For example, a pressure pulse wave may be used.

In the first embodiment, vascular endothelial function is evaluated based on the ratio of the mean values of the peripheral blood pressure index during the two periods, the anterior period ET₁ and the posterior period ET₂, obtained by dividing the evaluation duration ET (FIG. 10A). However, other methods may also be used to evaluate vascular endothelial function. For example, vascular endothelial function may be evaluated based on multiple calculated values of the peripheral blood pressure indices within the evaluation duration ET. For example, vascular endothelial function may be evaluated based on the trend of temporal changes in multiple calculated values of the peripheral blood pressure indices within the evaluation duration ET.

In the first embodiment, a ring-shaped device that can be worn on the finger is used as the pulse wave measurement device 20 (FIG. 2A), but other shapes may be used. For example, a clip-type device that can be worn on the fingertip may be used. Moreover, for the pulse wave measurement device 20, a wristwatch or wristband-type device that can be worn on the wrist may also be used as an alternative to a finger-worn device.

In the first embodiment, the upper arm is pressurized for vascular occlusion, and the pulse wave is measured at the finger. However, the vascular occlusion site and the pulse wave measurement site are not limited to these examples. Preferably, a part of the body is pressurized for vascular occlusion and the pulse wave is measured at a site farther from the heart than the pressurization site.

In the first embodiment, the control terminal 30 (FIG. 1) controls the vascular occlusion and release of the cuff sphygmomanometer 50. However, an independent device that performs vascular occlusion and release without communicating with the control terminal 30 may be used in an alternative aspect. For example, a cuff sphygmomanometer that does not communicate with the control terminal 30 may be used, or an aneroid sphygmomanometer may be used. In this case, the user or another individual nearby may operate the cuff for pressurization and release. The pulse wave feature calculation unit 41 (FIG. 1) is configured to detect the time points of the start and release of vascular occlusion from the acquired pulse wave.

In the first embodiment, the pulse wave measurement device 20, the control terminal 30, and the server 40 divide various functions as illustrated in FIG. 1. However, the functions may be divided in other manners. For example, the functions of the pulse wave feature calculation unit 41 may be implemented by the control terminal 30. All functions of the server 40 may be implemented by the control terminal 30. Conversely, all functions of the control terminal 30 may be implemented by the server 40.

In the first embodiment, the peripheral blood pressure indices calculated from the pulse wave after the release of vascular occlusion is used to evaluate vascular endothelial function. However, instead of the peripheral blood pressure indices calculated from the pulse wave during the period ET₂ after the release of vascular occlusion, the peripheral blood pressure indices calculated from the pulse wave during a certain duration before vascular occlusion may be used. For example, instead of the mean value of the peripheral blood pressure index for the posterior period ET₂ of the evaluation duration ET illustrated in FIG. 10A, the mean value of the peripheral blood pressure index for a certain duration before vascular occlusion may be used. Specifically, the mean value of the peripheral blood pressure index for the period ET₁ may be compared to the mean value of the peripheral blood pressure index for a certain duration before vascular occlusion.

As described herein, vascular endothelial function can be evaluated using both the peripheral blood pressure indices calculated from the pulse wave during the periods ET₁ and ET₂ after the release of vascular occlusion and the peripheral blood pressure indices calculated from the pulse wave during a certain duration before vascular occlusion. For example, vascular endothelial function may be evaluated based on the mean value of the peripheral blood pressure index for a certain duration before vascular occlusion and the peripheral blood pressure index for the period ET₂, and the mean value of the peripheral blood pressure index for the period ET₁. For the mean value of the peripheral blood pressure index for a certain duration before vascular occlusion and the peripheral blood pressure index for the period ET₂, either a simple average or weighted average weighted by the time deviation from the period ET₁ may be used.

Instead of the mean value of the peripheral blood pressure index for a certain duration before vascular occlusion and the peripheral blood pressure index for the period ET₂, a representative value of the peripheral blood pressure index for a certain duration before vascular occlusion and the peripheral blood pressure index for the period ET₂ may be obtained based on an approximation straight line or curve calculated from the waveform of the peripheral blood pressure index for the certain duration before vascular occlusion and the waveform of the peripheral blood pressure index for the period ET₂. Vascular endothelial function may be evaluated based on this representative value and the mean value of the peripheral blood pressure index for the period ET₁ after the release of vascular occlusion. For example, a value at the midpoint of the approximation straight line or curve may be used as the representative value.

The peripheral blood pressure indices can fluctuate due to various factors such as exercise, sudden temperature changes, or stress or tension. Evaluating vascular endothelial function with reference to the peripheral blood pressure indices before vascular occlusion reduces the influence of fluctuations in the peripheral blood pressure indices caused by various factors, thereby increasing evaluation accuracy. For example, when the mean value of the peripheral blood pressure index for the period ET₂ illustrated in FIG. 10A significantly differs from the mean value of the peripheral blood pressure index before vascular occlusion, the user may be notified to measure the pulse wave again.

In the first embodiment, the control terminal 30 (FIG. 1) is configured to control the pulse wave measurement device 20 (FIG. 1) and the cuff sphygmomanometer 50 (FIG. 1). However, the pulse wave measurement device 20 and the cuff sphygmomanometer 50 can communicate directly with each other, allowing the pulse wave measurement device 20 to control the timing of pressurization and release using the cuff sphygmomanometer 50.

In an exemplary aspect, the pulse wave measurement device 20 can be configured to measure the pulse wave in synchronization with the timing of pressurization and release by the cuff sphygmomanometer 50. In this manner, blood pressure can be measured concurrently with pulse wave measurement. Because vascular occlusion starts after a certain duration elapses from the start of pressurization, the control terminal 30 acquires the timing information about pressurization and release from the pulse wave measurement device 20, making it easier to identify the timing of vascular occlusion and release.

### Second Exemplary Embodiment

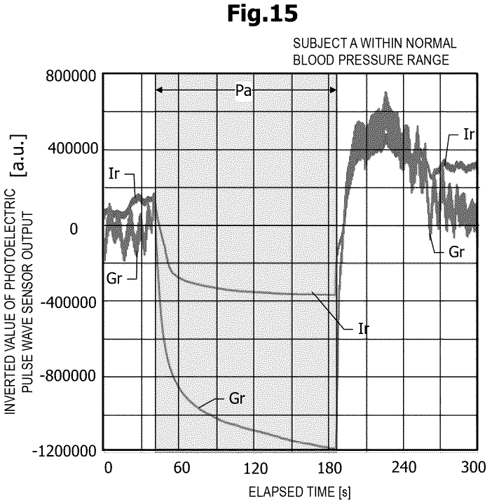

Next, a system and method for evaluating vascular endothelial function according to a second exemplary embodiment will be described with reference to the drawings in FIGS. 15 to 17. In the following, descriptions of the same configurational features as the system and method for evaluating vascular endothelial function according to the first embodiment described with reference to the drawings in FIGS. 1 to 14C are not repeated. In the first embodiment, the length (e.g., the vascular occlusion duration) of the vascular occlusion period Pa (FIGS. 9, 11, and 13) is approximately 30 seconds. In the second embodiment, the vascular occlusion duration is extended.

FIG. 15 is a graph illustrating the pulse wave measured with an extended vascular occlusion duration for subject A, whose pulse wave was measured as illustrated in

FIG. 9. The horizontal axis represents the elapsed time from the start of pulse wave measurement in units of [s], and the vertical axis represents the photoelectric pulse wave (the value obtained by inverting the output from the photoelectric pulse wave sensor 21 (FIG. 1)) in arbitrary units. To control the vascular occlusion duration, the cuff of an aneroid sphygmomanometer was used to pressurize the pressurization site. Solid lines Gr and Ir in the graph in FIG. 15 respectively illustrate the measurement results when the pulse wave is measured using green light and near-infrared light.

Approximately 30 seconds after the start of pulse wave measurement, pressurization with the aneroid sphygmomanometer was initiated. It takes approximately ten seconds plus several seconds from the start of pressurization to the start of vascular occlusion. Once vascular occlusion starts, the pulse wave can no longer be acquired. Approximately 150 seconds after the start of pulse wave measurement, the air in the cuff was released. The period during which vascular occlusion is ongoing is labeled Pa. The vascular occlusion period is shaded light gray in FIG. 15. The measurement was terminated when approximately 300 seconds had elapsed from the start of the measurement.

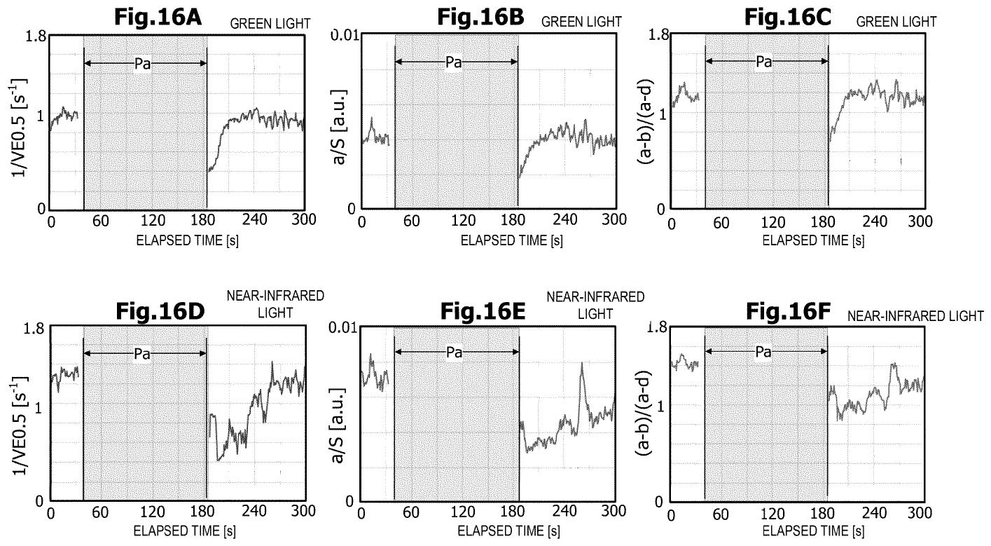

The graphs in FIGS. 16A to 16F illustrate the temporal changes in the peripheral blood pressure indices. The horizontal axis of these graphs represents the elapsed time in units of [s]. The vertical axis of the graphs in FIGS. 16A and 16D represents the peripheral blood pressure index “1/VE0.5” in units of [s⁻¹]. The vertical axis of the graphs in FIGS. 16B and 16E represents the peripheral blood pressure index “a/S” in arbitrary units. The vertical axis of the graphs in FIGS. 16C and 16F represents the peripheral blood pressure index “(a-b)/(a-d)”. The graphs in FIGS. 16A to 16C illustrate the peripheral blood pressure indices obtained from the pulse wave measured using green light. The graphs in FIGS. 16D to 16F illustrate the peripheral blood pressure indices obtained from the pulse wave measured using near-infrared light.

Comparing the graphs in FIGS. 10A to 10F, measured under a condition of relatively short vascular occlusion duration, with the graphs in FIGS. 16A to 16F, measured under a condition of relatively long vascular occlusion duration, reveals the following findings.

The phenomenon of the peripheral blood pressure indices decreasing upon the release of vascular occlusion, compared to before vascular occlusion, occurs regardless of the vascular occlusion duration. In the case of measurement under the condition of relatively short vascular occlusion duration, the peripheral blood pressure indices recover to the previous values after approximately 10 seconds. By contrast, in the case of measurement under the condition of relatively long vascular occlusion duration, it takes longer times for the peripheral blood pressure indices to return to the previous values. For example, in the case of measurement with green light, it takes approximately 30 seconds for the peripheral blood pressure indices to return to the previous values, and in the case of measurement with near-infrared light, it takes approximately 120 seconds for the peripheral blood pressure indices to return to the previous values. When the vascular occlusion duration is extended, the peripheral blood pressure indices in the case of measurement with near-infrared light indicate distinct decreases compared to when the vascular occlusion duration is relatively short.

The mechanism of the phenomenon of taking a longer time for the peripheral blood pressure indices to return to the previous values in the case of using near-infrared light can be considered as follows. When large blood vessels dilate upon the release of vascular occlusion, blood flow into the capillaries is inhibited. As a result, the peripheral blood pressure indices decrease. When the vascular occlusion duration is long, the blood flow rate in the capillaries and arterioles decreases, and it takes a longer time for blood to fill the capillaries. It also takes a longer time for blood to subsequently fill the arterioles.

In the pulse wave measurement using green light, information regarding blood flow in capillaries is primarily reflected in the pulse wave, whereas in the pulse wave measurement using near-infrared light, information regarding blood flow in arterioles as well as capillaries tends to be reflected in the pulse wave. Thus, the peripheral blood pressure indices measured using near-infrared light return to the previous values when blood fills the arterioles as well as the capillaries. As such, the time for the peripheral blood pressure indices measured using near-infrared light to recover to the previous values is longer than the time for the peripheral blood pressure indices measured using green light to recover to the previous values.

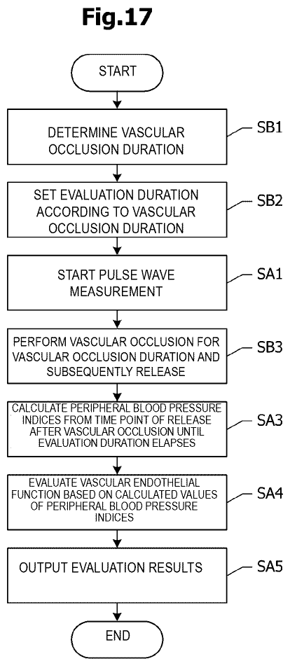

FIG. 17 is a flowchart illustrating the procedure for the method for evaluating vascular endothelial function according to the second exemplary embodiment. First, the user determines the vascular occlusion duration (step SB1). For example, the user inputs the determined vascular occlusion duration into the control terminal 30 (FIG. 1).

The control unit 31 of the control terminal 30 sets an evaluation duration according to the input vascular occlusion duration (step SB2). Subsequently, the control unit 31 starts pulse wave measurement (step SA1), as in the first embodiment (FIG. 3).

In the first embodiment (FIG. 3), vascular occlusion and release are performed according to a fixed vascular occlusion duration (step SA2), whereas in the second embodiment, vascular occlusion and release are performed according to the vascular occlusion duration that is set in step SB2 (step SB3). The following procedure from steps SA3 to SA5 is identical to the procedure from steps SA3 to SA5 in the first embodiment (FIG. 3).

Next, advantageous effects of the second embodiment are described.

In the second embodiment, by extending the vascular occlusion duration compared to the first embodiment, the peripheral blood pressure indices indicate distinct decreases after the release of vascular occlusion, thereby increasing the accuracy of vascular endothelial function evaluation. In addition, by extending the evaluation duration to match the extended vascular occlusion duration, changes in the decrease and recovery of the peripheral blood pressure indices after the release of vascular occlusion can be detected in a stable manner.

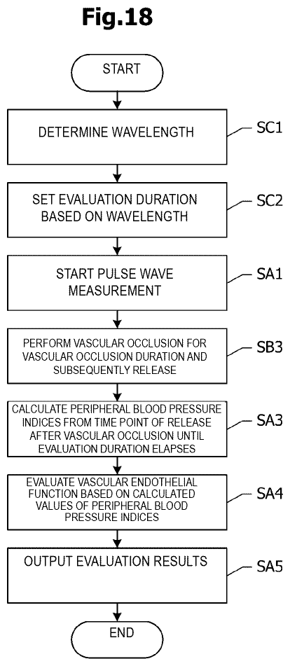

Next, a method for evaluating vascular endothelial function according to a modification of the second exemplary embodiment is described with reference to FIG. 18. FIG. 18 is a flowchart illustrating the procedure for the method for evaluating vascular endothelial function according to the modification of the second embodiment.

In the second embodiment, the user determines the vascular occlusion duration (step SB1), whereas in this modification, the user determines the wavelength of light used to measure the pulse wave (step SC1). Subsequently, the evaluation duration is set based on to the wavelength (step SC2). The following procedure is the same as in the second embodiment.

As illustrated in the graphs in FIGS. 16A to 16F, the peripheral blood pressure indices take a longer time to recover to the previous values when near-infrared light is used to measure the pulse wave compared to when green light is used. For this reason, the evaluation duration is preferably extended when near-infrared light is used compared to when green light is used. In this modification, because the evaluation duration is determined based on the wavelength of light used to measure the pulse wave, changes in the decrease and recovery of the peripheral blood pressure indices after the release of vascular occlusion can be detected in a stable manner.

### Third Exemplary Embodiment

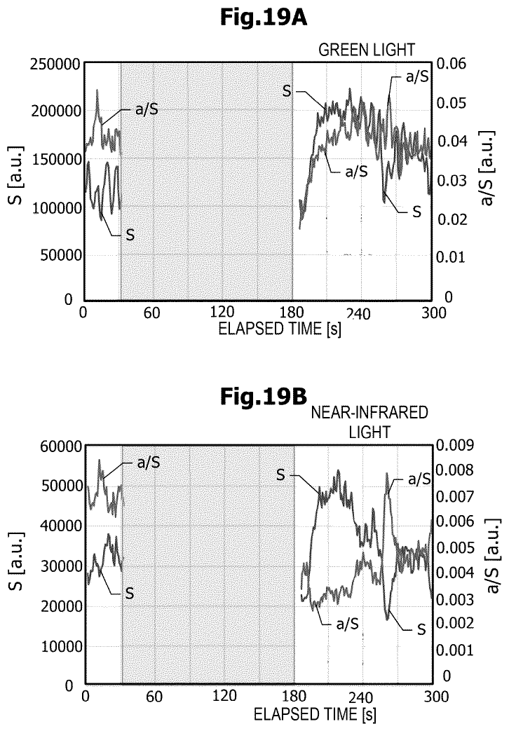

Next, a system and method for evaluating vascular endothelial function according to a third exemplary embodiment will be described with reference to the drawings in FIGS. 19A and 19B. In the following, descriptions of the same configurational features as the systems and methods for evaluating vascular endothelial function according to the first and second embodiments are not repeated.

FIGS. 19A and 19B are graphs illustrating the temporal changes in the amplitude S (FIG. 5) and the peripheral blood pressure index “a/S” obtained from the pulse wave of subject A illustrated in FIG. 15. The graph in FIG. 19A is obtained from the pulse waves measured using green light, whereas the graph in FIG. 19B is obtained from the pulse wave measured using near-infrared light.

It is shown that the amplitude S of the pulse wave is clearly larger during the period from the time point of vascular occlusion release until 60 seconds have elapsed, compared to the amplitude S of the pulse wave before vascular occlusion. The trend of the amplitude S of the pulse wave becoming larger appears prominently both when the measurement light is green light and when the measurement light is near-infrared light. It is assumed that the amplitude S of the pulse wave becomes larger due to the rise in blood flow rate caused by vasodilation.

When vascular endothelial function is optimal, blood vessels adequately dilate after the release of vascular occlusion, and the width of the increase in the amplitude S of the pulse wave becomes larger. When vascular endothelial function is impaired, blood vessels dilate insufficiently, and as a result, the width of the increase in the amplitude S of the pulse wave becomes smaller. As described above, vascular endothelial function can be evaluated based on the width of the increase in the amplitude S of the pulse wave after the release of vascular occlusion. In the third embodiment, vascular endothelial function is evaluated based on the width of the increase in the amplitude S of the pulse wave, in addition to the calculated values of the peripheral blood pressure indices from the time point of vascular occlusion release.

Next, advantageous effects of the third embodiment are described.

In the third embodiment, vascular endothelial function is evaluated based on the width of the increase in the amplitude S of the pulse wave, in addition to the calculated values of the peripheral blood pressure indices from the time point of vascular occlusion release, thereby increasing evaluation accuracy.

### Fourth Exemplary Embodiment

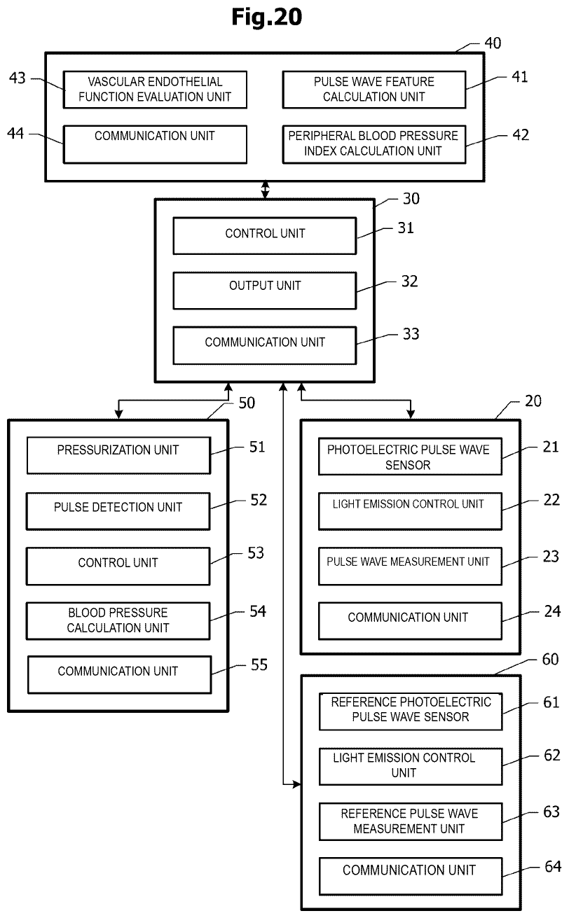

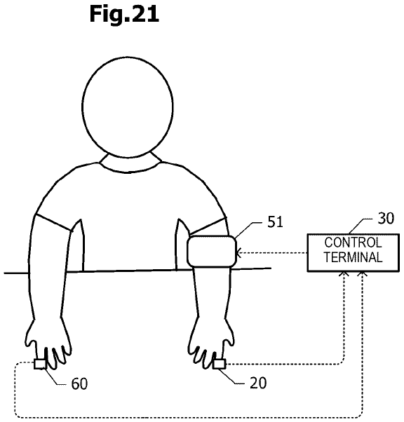

Next, a system and method for evaluating vascular endothelial function according to a fourth embodiment will be described with reference to the drawings in FIGS. 20 and 21. In the following, descriptions of the same configurational features as the systems and methods for evaluating vascular endothelial function according to the first embodiment are not repeated.

FIG. 20 is a block diagram of the system for evaluating vascular endothelial function according to the fourth embodiment. The system for evaluating vascular endothelial function according to the first embodiment includes a pulse wave measurement device 20, a control terminal 30, a server 40, and a cuff sphygmomanometer 50. By contrast, the system for evaluating vascular endothelial function according to the fourth embodiment additionally includes a reference pulse wave measurement device 60.

The reference pulse wave measurement device 60 includes a reference photoelectric pulse wave sensor 61, a light emission control unit 62, a reference pulse wave measurement unit 63, and a communication unit 64. The configuration and function of the reference photoelectric pulse wave sensor 61, the light emission control unit 62, the reference pulse wave measurement unit 63, and the communication unit 64 are identical to the configuration and function of the photoelectric pulse wave sensor 21, the light emission control unit 22, the pulse wave measurement unit 23, and the communication unit 24 of the pulse wave measurement device 20 according to the first embodiment. The reference pulse wave measurement unit 63 is configured to generate a reference pulse wave signal based on the output from the reference photoelectric pulse wave sensor 61. When a pulse wave sensor of a different method is used instead of the photoelectric pulse wave sensor 21 of the pulse wave measurement device 20, a reference pulse wave sensor designed to measure pulse waves using the same method as the photoelectric pulse wave sensor 21 is used instead of the reference photoelectric pulse wave sensor 61.

FIG. 21 is a schematic diagram of a user wearing a measurement device to evaluate vascular endothelial function using the system for evaluating vascular endothelial function according to the fourth embodiment. The user wraps a pressurization unit 51, such as a cuff, of the cuff sphygmomanometer 50 around one of the upper arms. The pulse wave measurement device 20 is worn on a finger of the same arm on which the pressurization unit 51 is worn. The reference pulse wave measurement device 60 is worn on a finger of the opposite arm. As such, the reference pulse wave measurement device 60 is attached to a site on the human body, with this site and the attachment site of the pulse wave measurement device 20 positioned symmetrically on the left and right sides.

Once pulse wave measurement starts, the pulse wave measurement device 20 acquires the pulse wave, while the reference pulse wave measurement device 60 acquires the reference pulse wave. The peripheral blood pressure indices calculated from the pulse wave acquired by the pulse wave measurement device 20 are compared with the peripheral blood pressure indices calculated from the reference pulse wave to evaluate vascular endothelial function.

The mean value of the peripheral blood pressure index during the period ET₁ (FIG. 10A) calculated from the pulse wave acquired by the pulse wave measurement device 20 is referred to as M₁, and the mean value during the period ET₂ is referred to as M₂. The mean value of the peripheral blood pressure index during the period ET₁ (FIG. 10A) calculated from the reference pulse wave is referred to as MR₁, and the mean value during the period ET₂ is referred to as MR₂. In the first embodiment, vascular endothelial function is evaluated based on M₁/M₂. However, in the fourth embodiment, vascular endothelial function is evaluated based on (M₁/M₂)/(MR₁/MR₂).

Next, advantageous effects of the fourth embodiment are described.

Because vascular occlusion is not performed on the finger on which the reference pulse wave is acquired, the mean values MR₁ and MR₂ are approximately equal. However, external factors may cause the mean value MR₂ to differ from the mean value MR₁. In other words, MR₁/MR₂ can deviate from 1. The influence of external factors is reflected to the same extent in the value of M₁/M₂. Since in the fourth embodiment vascular endothelial function is evaluated based on (M₁/M₂)/(MR₁/MR₂), the influence of external factors can be almost eliminated, thereby increasing the accuracy of vascular endothelial function evaluation.

The aforementioned embodiments are illustrative, and partial replacement or combination of the configuration elements presented in different embodiments is possible. The same effects and advantages of the same configurational features among multiple embodiments are not described in every embodiment. It is generally note that the exemplary aspects of the present disclosure are not limited to the aforementioned embodiments. For example, various modifications, improvements, and combinations would be readily apparent to those skilled in the art.

### REFERENCE SIGNS LIST

20 pulse wave measurement device
21 photoelectric pulse wave sensor
21A, 21B light-emitting element
21C light-receiving element
22 light emission control unit
23 pulse wave measurement unit
24 communication unit
27 wearable member
30 control terminal
31 control unit
32 output unit
33 communication unit
40 server
41 pulse wave feature calculation unit
42 peripheral blood pressure index calculation unit
43 vascular endothelial function evaluation unit
44 communication unit
50 cuff sphygmomanometer
51 pressurization unit
52 pulse detection unit
53 control unit
54 blood pressure calculation unit
55 communication unit
60 reference pulse wave measurement device
61 reference photoelectric pulse wave sensor
62 light emission control unit
63 reference pulse wave measurement unit
64 communication unit
70 user's body surface
71 epidermal region
72 arterioles
73 capillaries

1. A system for evaluating vascular endothelial function, comprising:
a photoelectric pulse wave sensor including a light-receiving element that is configured to detect light emitted by a light-emitting element and that passes through biological tissue of a user, the photoelectric pulse wave sensor being configured to attach to a site farther from a heart of the user than a pressurization site of the user that is pressurized for vascular occlusion;
a pulse wave measurement unit configured to generate a pulse wave signal based on the light detected by the light-receiving element;
a peripheral blood pressure index calculation unit configured to calculate a peripheral blood pressure index based on a steepness of a rise per beat of the pulse wave signal generated by the pulse wave measurement unit;
a vascular endothelial function evaluation unit configured to evaluate a vascular endothelial function based on a calculated value of the calculated peripheral blood pressure index from a time point of vascular occlusion release until an evaluation duration elapses; and
an output unit configured to generate a display relating to the vascular endothelial function.

2. The system for evaluating vascular endothelial function according to claim 1, wherein the vascular endothelial function evaluation unit is configured to evaluate the vascular endothelial function based on both a value of the calculated peripheral blood pressure index before vascular occlusion and a value of the calculated peripheral blood pressure index after vascular occlusion release.

3. The system for evaluating vascular endothelial function according to claim 1, further comprising a ring-shaped wearable member configured to be worn on a finger and that includes the photoelectric pulse wave sensor.

4. The system for evaluating vascular endothelial function according to claim 1, further comprising a cuff sphygmomanometer that is configured to pressurize the pressurization site of the user.

5. The system for evaluating vascular endothelial function according to claim 4, wherein:
the cuff sphygmomanometer and the pulse wave measurement unit are configured to wirelessly communicate with each other,
the cuff sphygmomanometer is configured to notify the pulse wave measurement unit of a timing of vascular occlusion and release, and
the pulse wave measurement unit is configured to calculate the peripheral blood pressure index in synchronization with the timing of vascular occlusion and release of the cuff sphygmomanometer.

6. The system for evaluating vascular endothelial function according to claim 4, further comprising a control terminal that includes a processor configured to execute instructions on an electronic memory to:
wirelessly communicate with the cuff sphygmomanometer and the pulse wave measurement unit,
control an operation for vascular occlusion and release of the cuff sphygmomanometer, and
receive the pulse wave signal from the pulse wave measurement unit and transmit the pulse wave signal to the peripheral blood pressure index calculation unit.

7. The system for evaluating vascular endothelial function according to claim 1, wherein the peripheral blood pressure index calculation unit is configured to calculate the peripheral blood pressure index to include information regarding a peak value of wave a of an acceleration pulse wave obtained by calculating a second derivative of a waveform of the pulse wave signal generated by the pulse wave measurement unit, and an amplitude of the waveform of the pulse wave signal generated by the pulse wave measurement unit.

8. The system for evaluating vascular endothelial function according to claim 1, wherein the peripheral blood pressure index calculation unit is configured to calculate the peripheral blood pressure index to include information regarding a difference between a peak value of wave a of an acceleration pulse wave obtained by calculating a second derivative of a waveform of the pulse wave signal generated by the pulse wave measurement unit and a peak value of wave d of the acceleration pulse wave, and a difference between the peak value of wave a and a peak value of wave b of the acceleration pulse wave.

9. The system for evaluating vascular endothelial function according to claim 1, wherein the peripheral blood pressure index calculation unit is configured to calculate the calculate peripheral blood pressure index to include information regarding a width of a first peak that appears within one beat of a velocity pulse wave obtained by calculating a first derivative of a waveform of the pulse wave signal generated by the pulse wave measurement unit.

10. The system for evaluating vascular endothelial function according to claim 1, wherein the vascular endothelial function evaluation unit is configured to evaluate the vascular endothelial function based additionally on a temporal change in an amplitude of the pulse wave signal generated by the pulse wave measurement unit from the time point of vascular occlusion release until a certain duration elapses.

11. The system for evaluating vascular endothelial function according to claim 1, wherein the photoelectric pulse wave sensor includes the light-emitting element and is configured to emit light in a wavelength range from blue to yellow-green.

12. The system for evaluating vascular endothelial function according to claim 11, wherein the photoelectric pulse wave sensor is configured such that the light-emitting element is separated by a distance from the light-receiving element that is greater than or equal to 1 mm and less than or equal to 3 mm.

13. The system for evaluating vascular endothelial function according to claim 1, wherein the vascular endothelial function evaluation unit is configured to change the evaluation duration based on a vascular occlusion duration.

14. The system for evaluating vascular endothelial function according to claim 13, wherein:
the light-emitting element of the photoelectric pulse wave sensor is configured to emit light at least two different wavelengths, and
the vascular endothelial function evaluation unit is configured to change the evaluation duration based on a wavelength of light used to acquire the pulse wave signal.

15. The system for evaluating vascular endothelial function according to claim 1, further comprising:
a reference pulse wave measurement unit configured to generate a reference pulse wave signal based on a measurement result from a reference pulse wave sensor attached to a site that is positioned symmetrically on the user to an attachment site of the photoelectric pulse wave sensor.

16. The system for evaluating vascular endothelial function according to claim 15, wherein the vascular endothelial function evaluation unit is configured to evaluate the vascular endothelial function based additionally on a peripheral blood pressure index calculated based on the reference pulse wave signal generated by the reference pulse wave measurement unit.

17. An apparatus for evaluating vascular endothelial function, comprising:
a pulse wave measurement device configured to generate a pulse wave signal based on a measurement result from a pulse wave sensor attached to a site of a user that is farther from a heart of the user than a pressurization site that is pressurized for vascular occlusion, the pulse wave sensor including a light-receiving element that is configured to detect light emitted by a light-emitting element and that passes through biological tissue of the user for the measurement result; and
a control terminal that includes a processor configured to execute instructions on an electronic memory to:
calculate a peripheral blood pressure index based on a steepness of a rise per beat of the pulse wave signal generated by the pulse wave measurement device,
evaluate a vascular endothelial function based on a value of the calculated peripheral blood pressure index from a time point of vascular occlusion release until an evaluation duration elapses, and
output an evaluation result as a display relating to the evaluated vascular endothelial function.

18. The apparatus for evaluating vascular endothelial function according to claim 17, wherein:
the pulse wave sensor includes a light-emitting element configured to emit the light in a wavelength range from blue to yellow-green, and
the pulse wave sensor is configured such that the light-emitting element is separate by a distance from the light-receiving element that is greater than or equal to 1 mm and less than or equal to 3 mm.

19. The apparatus for evaluating vascular endothelial function according to claim 18, wherein:
the light-emitting element of the pulse wave sensor is configured to emit light at least two different wavelengths, and
the control unit is further configured to change the evaluation duration based on a wavelength of light used to acquire the pulse wave signal.

20. A method for evaluating vascular endothelial function, the method comprising:
performing vascular occlusion by pressurizing a part of a body of a user and subsequently releasing vascular occlusion;
acquiring a pulse wave signal based on light, detected by a light-receiving element, that passes through an arteriole or capillary at a site farther from a heart of the user than a pressurized site from a time point of vascular occlusion release until a certain duration elapses;
obtaining a temporal change in a peripheral blood pressure index based on a steepness of a rise per beat of the acquired pulse wave signal;
evaluating a vascular endothelial function based on the temporal change in the peripheral blood pressure index; and
generating, by an output, a display relating to the evaluated vascular endothelial function.
